SEQUENCE LISTING (18 sequences)

SEQ ID NO: 1 - DNA, 38 bp, synthetic construct
  source 1..38: mol_type = other DNA
tgtaaaacgacggccagtttgcagagcaagcaagcaag

SEQ ID NO: 2 - DNA, 20 bp, synthetic construct
  source 1..20: mol_type = other DNA
agaaatgaggcaggaacaca

SEQ ID NO: 3 - DNA, 229 bp, synthetic construct
  source 1..229: mol_type = other DNA
ttgcagagcaagcaagcaagtagcagacaagtaaaccttgaacaagacttgcaaatcactatcacacaaagcaactactactactacaaatcactaatgaacaagaagaagaagaagtagaacaacaagtctatgaagaagcacacagttactgttatgaccttatctggataccatatatttctctagactttagaatatatgtataatgtgttcctgcctcatttct

SEQ ID NO: 4 - DNA, 232 bp, synthetic construct
  source 1..232: mol_type = other DNA
ttgcagagcaagcaagcaagtagcagacaagtaaaccttgaacaagacttgcaaatcactatcacacaaagcaactactactactacaaatcactaatgaacaagaagaagaagaagaagtagaacaacaagtctatgaagaagcacacagttactgttatgaccttatctggataccatatatttctctagactttagaatatatgtataatgtgttcctgcctcatttct

SEQ ID NO: 5 - DNA, 38 bp, synthetic construct
  source 1..38: mol_type = other DNA
tgtaaaacgacggccagtagttggagttgtctcagcgg

SEQ ID NO: 6 - DNA, 20 bp, synthetic construct
  source 1..20: mol_type = other DNA
aggaggaaagggaacgcttg

SEQ ID NO: 7 - DNA, 218 bp, synthetic construct
  source 1..218: mol_type = other DNA
agttggagttgtctcagcggcggccattgctttcgcaacctcggctttctgctcctcctcgatacgaagtcgttcacgttcctcctcctcctcctcctccttcagcctctcctgctccttggcctgctcttctgcgagacgaacacgttctttctcttcgtcttcaagcatacgtatacgttcttcctcttctttgcgcaagcgttccctttcctcct

SEQ ID NO: 8 - DNA, 221 bp, synthetic construct
  source 1..221: mol_type = other DNA
agttggagttgtctcagcggcggccattgctttcgcaacctcggctttctgctcctcctcgatacgaagtcgttcacgttcctcctcctcctcctcctcctccttcagcctctcctgctccttggcctgctcttctgcgagacgaacacgttctttctcttcgtcttcaagcatacgtatacgttcttcctcttctttgcgcaagcgttccctttcctcct

SEQ ID NO: 9 - DNA, 38 bp, synthetic construct
  source 1..38: mol_type = other DNA
tgtaaaacgacggccagtctcgctagcgcatcgtgata

SEQ ID NO: 10 - DNA, 20 bp, synthetic construct
  source 1..20: mol_type = other DNA
ggccggcttatccctaaaca

SEQ ID NO: 11 - DNA, 231 bp, synthetic construct
  source 1..231: mol_type = other DNA
ctcgctagcgcatcgtgataagaaaagagagagcttagaaaagaggaaaagtgacaaacggaatgtaatggtcgtgttcggataaagtagagcatataccttccccacaccagcctcttaaaactagtagtactacacatcgcatgtaaagcgccccaatgaccgcgtttctggatttaaatatatacatatatatatatataaatggtcgtgtttagggataagccggcc

SEQ ID NO: 12 - DNA, 233 bp, synthetic construct
  source 1..233: mol_type = other DNA
ctcgctagcgcatcgtgataagaaaagagagagcttagaaaagaggaaaagtgacaaacggaatgtaatggtcgtgttcggataaagtagagcatataccttccccacaccagcctcttaaaactagtagtactacacatcgcatgtaaagcgccccaatgaccgcgtttctggatttaaatatatacatatatatatatatataaatggtcgtgtttagggataagccggcc

SEQ ID NO: 13 - DNA, 235 bp, synthetic construct
  source 1..235: mol_type = other DNA
ctcgctagcgcatcgtgataagaaaagagagagcttagaaaagaggaaaagtgacaaacggaatgtaatggtcgtgttcggataaagtagagcatataccttccccacaccagcctcttaaaactagtagtactacacatcgcatgtaaagcgccccaatgaccgcgtttctggatttaaatatatacatatatatatatatatataaatggtcgtgtttagggataagccggcc

SEQ ID NO: 14 - DNA, 237 bp, synthetic construct
  source 1..237: mol_type = other DNA
ctcgctagcgcatcgtgataagaaaagagagagcttagaaaagaggaaaagtgacaaacggaatgtaatggtcgtgttcggataaagtagagcatataccttccccacaccagcctcttaaaactagtagtactacacatcgcatgtaaagcgccccaatgaccgcgtttctggatttaaatatatacatatatatatatatatatataaatggtcgtgtttagggataagccggcc

SEQ ID NO: 15 - DNA, 38 bp, synthetic construct
  source 1..38: mol_type = other DNA
tgtaaaacgacggccagtggaagatgagccagctcctg

SEQ ID NO: 16 - DNA, 20 bp, synthetic construct
  source 1..20: mol_type = other DNA
agatgggctctttggacgtg

SEQ ID NO: 17 - DNA, 212 bp, synthetic construct
  source 1..212: mol_type = other DNA
ggaagatgagccagctcctgctgtcactgaaccagaggctgctgctgctgctactactaccactgagaccccagctgccaccgaggaaccttctaaggaggaagctaaacctgtatgtataccctagcttttgtgttaacaacactaattcttttatgttataggctgccgattctgacaatggtaaggctgcacgtccaaagagcccatct

SEQ ID NO: 18 - DNA, 215 bp, synthetic construct
  source 1..215: mol_type = other DNA
ggaagatgagccagctcctgctgtcactgaaccagaggctgctgctgctgctgctactactaccactgagaccccagctgccaccgaggaaccttctaaggaggaagctaaacctgtatgtataccctagcttttgtgttaacaacactaattcttttatgttataggctgccgattctgacaatggtaaggctgcacgtccaaagagcccatct

(19) United States
(12) Patent Application Publication
(10) Pub. No.: US 2024/0218462 A1
(43) Pub. Date: Jul. 4, 2024

(54) DNA BARCODE FOR SCREENING TOTAL POLYPHENOL CONTENT INDEX OF FLOCCULARIA LUTEOVIRENS

(71) Applicant: XIZANG VOCATIONAL TECHNICAL COLLEGE, LHASA (CN)

(72) Inventors: MANJUN YANG, LHASA (CN); CAIXIA ZHANG, LHASA (CN); YISHUI CAI, LHASA (CN)

(21) Appl. No.: 18/417,255

(22) Filed: Jan. 19, 2024

Related U.S. Application Data
(63) Continuation of application No. PCT/CN2022/109894, filed on Aug. 3, 2022.

(30) Foreign Application Priority Data
Nov. 19, 2021 (CN) 202111399047.6

(51) Int. Cl. C12Q 1/6895; C12Q 1/686

(52) U.S. Cl. CPC C12Q 1/6895; C12Q 1/686

(57) ABSTRACT

The present invention discloses a DNA barcode, a primer group and application for screening a total polyphenol content index of Floccularia luteovirens, belonging to the technical field of screening of edible fungi germplasm resources. Compared with a traditional breeding method and other existing DNA barcode technologies, the present invention has the advantages of time saving, labor saving, money saving, accuracy and high efficiency, plays a positive role in original place identification and genetic breeding of high-quality Floccularia luteovirens, and also provides an effective method for identification and protection of germplasm resources.

### SEQUENCE LISTING

The instant application contains a Sequence Listing which has been submitted electronically in ST.26 format and is hereby incorporated by reference in its entirety. Said ST.26 copy, created on Jan. 18, 2024, is named Sequence Listing.xml and is 18,885 bytes in size.

### TECHNICAL FIELD

The present invention relates to the technical field of screening of edible fungi germplasm resources, in particular to a DNA barcode, a primer group and application for screening a total polyphenol content index of Floccularia luteovirens.

### BACKGROUND

Floccularia luteovirens in a gold yellow color, also known as yellow mushroom and golden mushroom, is a high-quality edible fungus with a unique flavor, which cannot be cultivated artificially at present. Wild Floccularia luteovirens is mainly distributed in Qinghai-Tibet Plateau; and the main producing areas include Dangxiong County of Tibet Autonomous Region, Qilian County of Qinghai Province and Shiqu County of Sichuan Province, and the quality of these three main producing areas is the best. Main indexes to evaluate the nutritional value, flavor and biological activity of Floccularia luteovirens include: high contents and strong antioxidant activity of total soluble proteins, total soluble amino acids, total polyphenols, total polysaccharides and total fat. In different producing places, Floccularia luteovirens has different nutritional values, different flavors, different biological activities and different market prices. In the past, the breeding of Floccularia luteovirens was mainly determined by a morphological method combined with beneficial content indexes. However, due to the influence of the special climate environment of Qinghai-Tibet Plateau, the phenomena of different objects with the same name and the same object with different names often appeared to Floccularia luteovirens produced in different areas, so morphological identification was difficult to realize effective distinguishment. More difficultly, it is impossible to screen out high-quality strains with high contents of total soluble proteins, total soluble amino acids, total polyphenols, total polysaccharides and total fat, and strong antioxidant activity by the morphological method. In addition, due to high altitude of main producing areas thereof, it is also difficult to collect samples.

A DNA barcode molecular identification technology is a molecular biology technology based on DNA barcodes (conserved and stable genetic DNA sequences in a genome) to recognize and identify species and excellent quality. It is the effective supplement and expansion of traditional breeding methods, and can accurately and effectively identify samples when samples are incomplete in morphologies or lack morphological structures (processed products such as powder, etc.). In order to realize effective development and utilization of Floccularia luteovirens, it is particularly important and urgent to screen different producing areas of Floccularia luteovirens strains with assistance of the DNA barcode molecular identification technology. In the existing DNA barcode technology, non-coding regions or conserved gene sequences in ITS (internal transcribed spacer in ribosomal RNA) and mitochondria are mainly used for species object identification; the operation of restriction fragment length polymorphism (RFLP) is very complicated; reliability and repeatability of results are poor; random amplified polymorphic DNA (RAPD) is easily disturbed, which requires a high technical level of operators and is difficult to popularize in assisted breeding; and single nucleotide polymorphism (SNP) has high requirements for equipment and high cost.

Therefore, in view of shortcomings of traditional breeding methods that the breeding of Floccularia luteovirens strains is not accurate enough, time-consuming and labor-consuming, an urgent problem to be solved by those skilled in the art is how to provide a DNA barcode that can accurately and quickly identify the strains of Floccularia luteovirens and realize high-quality breeding, which has the characteristics of low cost, high efficiency, simple operation, stable and reliable results and good repeatability.

### SUMMARY

In view of this, the present invention provides a DNA barcode and a primer group for screening a total polyphenol content index of Floccularia luteovirens, which can quickly and accurately screen out strains with a high total polyphenol content of Floccularia luteovirens, and provides a favorable auxiliary means for breeding high-quality Floccularia luteovirens.

In order to achieve the above purpose, the present invention adopts the following technical solution:

A DNA barcode for screening a total polyphenol content index of Floccularia luteovirens, wherein a nucleotide sequence of the DNA barcode includes one or more of:
SEQ ID NO:3,
and/or SEQ ID NO:4,
and/or a combination of SEQ ID NO:3 and SEQ ID NO:4,
and SEQ ID NO:7,
and/or SEQ ID NO:8,
and/or a combination of SEQ ID NO:7 and SEQ ID NO:8,
and/or a combination of SEQ ID NO:11 and SEQ ID NO:12 and SEQ ID NO:13,
and/or a combination of SEQ ID NO:12 and SEQ ID NO:13 and SEQ ID NO:14,
and/or a combination of SEQ ID NO:13 and SEQ ID NO:14,
and/or SEQ ID NO:17,
and/or SEQ ID NO:18,
and/or a combination of SEQ ID NO:17 and SEQ ID NO:18.

According to the present invention, based on all simple sequence repeats (SSR) in the whole genome of Floccularia luteovirens, fluorescent PCR amplification is carried out; and a DNA barcode which effectively corresponds to a total polyphenol content is established. Compared with the DNA barcode of the present invention, the fragment obtained by amplification can quickly and accurately screen out strains with a high total polyphenol content of Floccularia luteovirens, which provides favorable assistance for breeding of Floccularia luteovirens.

Another purpose of the present invention is to provide a primer group for amplifying the DNA barcode for screening the total polyphenol content index of Floccularia luteovirens, wherein a nucleotide sequence of the primer group includes one or more groups of:
SEQ ID NO: 1 and SEQ ID NO:2,
and/or SEQ ID NO:5 and SEQ ID NO:6,
and/or SEQ ID NO:9 and SEQ ID NO: 10,
and/or SEQ ID NO:15 and SEQ ID NO:16.

As a preferred technical solution of the present invention, the nucleotide sequence of the primer group includes:
such as SEQ ID NO:1 and SEQ ID NO:2,
and SEQ ID NO:5 and SEQ ID NO:6,
and SEQ ID NO:9 and SEQ ID NO:10,
and SEQ ID NO:15 and SEQ ID NO:16.

Different primer groups of the present invention can be used alone or in combination to screen the total polyphenol content of Floccularia luteovirens; and when all primer groups are used together, the screening accuracy is the highest.

Another purpose of the present invention is to provide a method for screening Floccularia luteovirens by using the total polyphenol content index, which comprises the following steps:
S1, extracting genomic DNA of a sample to be tested;
S2, taking the genomic DNA in S1 as a template, and performing a fluorescent PCR amplification reaction on one or more of the above primer groups to obtain an amplification product;
S3, detecting the amplification product of S2 by capillary fluorescence electrophoresis, and judging by the number of fragments, the number of SSR loci, SSR repetitive elements and repeating times of the amplification product.

As a preferred technical solution of the present invention, a judgment standard in step S3 is:
when the primer group of SEQ ID NO:1 and SEQ ID NO:2 is amplified to obtain a 229 bp fragment containing 5 AAG repetitive elements and a 232 bp fragment containing 6 AAG repetitive elements;
and/or the primer group of SEQ ID NO:5 and SEQ ID NO:6 is amplified to obtain a 218 bp fragment containing 7 TCC repetitive elements and a 221 bp fragment containing 8 TCC repetitive elements;
and/or the primer group of SEQ ID NO:9 and SEQ ID NO:10 is amplified to obtain a 233 bp fragment containing 8 AT repetitive elements, a 235 bp fragment containing 9 AT repetitive elements, and a 237 bp fragment containing 10 AT repetitive elements;
and/or the primer group of SEQ ID NO:15 and SEQ ID NO:16 is amplified to obtain a 212 bp fragment containing 5 GCT repetitive elements and a 215 bp fragment containing 6 GCT repetitive elements the Floccularia luteovirens is determined as Floccularia luteovirens with high total polyphenol content.

As a preferred technical solution of the present invention, a reaction system of the fluorescent PCR amplification reaction in step S2 is:
5 μL of 2×Taq PCR Master Mix, 1 μL of genomic DNA, 0.1 μL of forward primer, 0.4 μL of reverse primer, and 0.4 μL of M13 primer with fluorescence, wherein a volume is fixed to 10 μL with sterile deionized water.

Further preferably, the concentrations of the forward primer, the reverse primer and the M13 primer with fluorescence are all 10 uM.

As a preferred technical solution of the present invention, a fluorescent PCR amplification reaction procedure in step S2 is:
performing pre-denaturation at 95° C. for 3 min, denaturation at 95° C. for 30 s, PCR annealing during the decrease from 62° C. to 55° C. for 30 s, and extension at 72° C. for 30 s, with a total of 10 cycles; performing denaturation at 95° C. for 30 s, annealing at 52° C. for 30 s, and extension at 72° C. for 30 s, with a total of 25 cycles; performing final extension at 72° C. for 20 min; and after heat preservation at 4° C. for 6 h, using the product for fluorescence capillary electrophoresis detection.

Further another purpose of the present invention is to provide application of the DNA barcode and/or the primer group in preparation of a product for screening high-quality Floccularia luteovirens with the total polyphenol content index.

Further another purpose of the present invention is to provide a product for screening high-quality Floccularia luteovirens with the total polyphenol content index, which comprises one or more of the above primer groups, and satisfies the standard that the primer group of SEQ ID NO:1 and SEQ ID NO:2 is amplified to obtain a 229 bp fragment containing 5 AAG repetitive elements and a 232 bp fragment containing 6 AAG repetitive elements;
and/or the primer group of SEQ ID NO:5 and SEQ ID NO:6 is amplified to obtain a 218 bp fragment containing 7 TCC repetitive elements and a 221 bp fragment containing 8 TCC repetitive elements;
and/or the primer group of SEQ ID NO:9 and SEQ ID NO:10 is amplified to obtain a 233 bp fragment containing 8 AT repetitive elements, a 235 bp fragment containing 9AT repetitive elements and a 237 bp fragment containing 10 AT repetitive elements;
and/or the primer group of SEQ ID NO:15 and SEQ ID NO:16 is amplified to obtain a 212 bp fragment containing 5 GCT repetitive elements and a 215 bp fragment containing 6 GCT repetitive elements.

As a preferred technical solution of the present invention, the product is a kit.

According to the above technical solution, compared with the prior art, the present invention discloses and provides the DNA barcode and the primer group for screening the total polyphenol content index, which can utilize wild samples of Floccularia luteovirens and a small amount of tissues or hyphae to carry out character breeding of high-quality strains; identification can be carried out at different growth stages such as mycelium, primordia, fruiting bodies and spores; and the present invention has the short detection period, simple operation, no waste, stable and reliable results and good repeatability, and overcomes the shortcomings of traditional breeding methods that the breeding of Floccularia luteovirens strains is not accurate enough, time-consuming and labor-consuming.

Compared with a traditional breeding method and other existing DNA barcode technologies, the present invention has the advantages of time saving, labor saving, money saving, accuracy and high efficiency, plays a positive role in character screening and genetic breeding of high-quality Floccularia luteovirens, and also provides an effective method for identification and protection of germplasm resources.

### DESCRIPTION OF DRAWINGS

To more clearly describe the technical solutions in the embodiments of the present invention or in the prior art, the drawings required to be used in the description of the embodiments or the prior art will be simply presented below. Apparently, the drawings in the following description are merely the embodiments of the present invention, and for those ordinary skilled in the art, other drawings can also be obtained according to the provided drawings without contributing creative labor.

FIG. 1 is a diagram of comparison results of total polyphenol contents of test example, reference example 1 and reference example 2 according to the present invention;

FIG. 2A is a diagram of reference example 1 of fluorescent PCR amplification using primer 1 according to the present invention;

FIG. 2B is a diagram of reference example 2 of fluorescent PCR amplification using primer 1 according to the present invention;

FIG. 2C is a diagram of test example of fluorescent PCR amplification using primer 1 according to the present invention;

FIG. 3A is a diagram of reference example 1 of fluorescent PCR amplification using primer 2 according to the present invention;

FIG. 3B is a diagram of reference example 2 of fluorescent PCR amplification using primer 2 according to the present invention;

FIG. 3C is a diagram of test example of fluorescent PCR amplification using primer 2 according to the present invention;

FIG. 4A is a diagram of reference example 1 of fluorescent PCR amplification using primer 3 according to the present invention;

FIG. 4B is a diagram of reference example 2 of fluorescent PCR amplification using primer 3 according to the present invention;

FIG. 4C is a diagram of test example of fluorescent PCR amplification using primer 3 according to the present invention;

FIG. 5A is a diagram of reference example 1 of fluorescent PCR amplification using primer 4 according to the present invention;

FIG. 5B is a diagram of reference example 2 of fluorescent PCR amplification using primer 4 according to the present invention; and

FIG. 5C is a diagram of test example of fluorescent PCR amplification using primer 4 according to the present invention.

### DETAILED DESCRIPTION

The technical solutions in the embodiments of the present invention will be clearly and fully described below in combination with the drawings in the embodiments of the present invention. Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

Embodiments of the present invention disclose a DNA barcode, a primer group and application for screening a total polyphenol content index of Floccularia luteovirens. Reagents used are commercially available; and sources thereof are not specifically limited. Unless otherwise mentioned, test methods used are conventional methods.

### Embodiment 1 Establishment of a DNA Barcode of Floccularia luteovirens

Samples of Floccularia luteovirens are collected from Dangxiong County of Tibet Autonomous Region, Qilian County of Qinghai Province and Shiqu County of Sichuan Province and are treated by genome sequencing; and SSR loci in the genome sequences are analyzed by MISA program.

Primers are designed for PCR amplification of the SSR loci; the primers that can amplify the corresponding fragments are reserved; and invalid primers are discarded.

The total polyphenol contents of Floccularia luteovirens samples from Dangxiong County of Tibet Autonomous Region, Qilian County of Qinghai Province and Shiqu County of Sichuan Province are determined.

The samples from the above three producing areas are amplified by effective primers and detected by capillary electrophoresis. The simple sequence repeat (SSR) locus corresponding to the total polyphenol content is established by analysis. Finally, four pairs of primers (see Table 1) are obtained. The four pairs of primers are used to amplify the sample genome; and the fragment polymorphism obtained can assist in screening Floccularia luteovirens with the high total polyphenol content.

TABLE 1
Specific primers for screening of high-quality strains of Floccularia
luteovirens with high total polyphenol contents

| | | Denaturation | | Denaturation | |
|---|---|---|---|---|---|
| | | temperature | | temperature | SSR |
| No. | Forward primer | (° C.) | Reverse primer | (° C.) | element |
| 1 | TGTAAAACGACGGC- | 59.968 | AGAAATGAG- | 57.043 | AAG |
| | CAGTTTGCAGAGCA- | | GCAGGAACA- | | |
| | AGCAAGCAAG, as | | CA, as shown in | | |
| | shown in SEQ ID No: 1 | | SEQ ID No: 2 | | |
| 2 | TGTAAAACGACGGC- | 59.966 | AGGAGGAAA- | 59.963 | TCC |
| | CAGTAGTTGGAGTTG- | | GGGAACGCT- | | |
| | TCTCAGCGG, as shown | | TG, as shown in | | |
| | in SEQ ID No: 5 | | SEQ ID No: 6 | | |
| 3 | TGTAAAACGACGGC- | 60.11 | GGCCGGCTT- | 59.818 | AT |
| | CAGTCTCGCTAGCGC- | | ATCCCTAAAC- | | |
| | ATCGTGATA, as shown | | A, as shown in | | |
| | in SEQ ID No: 9 | | SEQ ID No: 10 | | |
| 4 | TGTAAAACGACGGC- | 60.179 | AGATGGGCT- | 60.036 | GCT |
| | CAGTGGAAGATGAG- | | CTTTGGACGT- | | |
| | CCAGCTCCTG, as | | G, as shown in | | |
| | shown in SEQ ID No: 15 | | SEQ ID No: 16 | | |

### Embodiment 2 Amplification of SSR Specific Primers for Total Polyphenol Contents of Floccularia luteovirens Strains

### (1) Extraction of Total Polyphenols

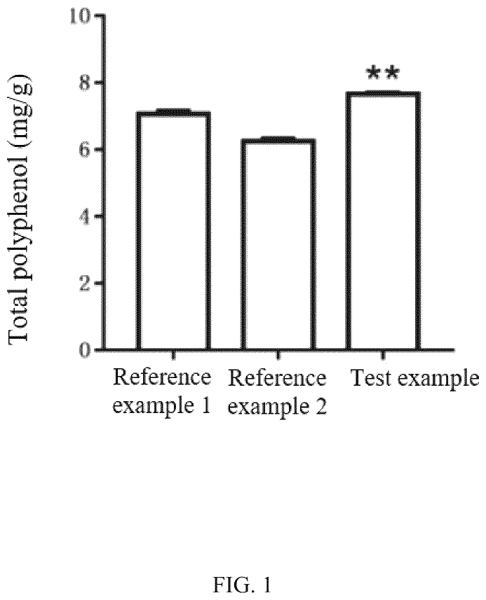

Fruiting bodies of Floccularia luteovirens are collected from Dangxiong County of Tibet Autonomous Region, Qilian County of Qinghai Province and Shiqu County of Sichuan Province, dehydrated by a vacuum freeze-drying method, smashed and sieved with a 50 mesh sieve; 1 g of dry powder is added with 20 mL of double distilled water, extracted with assistance of the 300 W ultrasonic wave for 30 min, and then centrifuged at 5000 r/min for 30 min; and the supernatant is taken to prepare a total polyphenol extracting solution. A total polyphenol content in the extract of Floccularia luteovirens fruiting bodies is determined by a Folin-phenol method. Please refer to Wang Jiadan et al. (Wang Jiadan, Xu Ting and Han Wei. Optimization of methods for determining a total polyphenol content in Flammulina velutipes [J]. Journal of Nanjing University of Technology (Version of Natural Science), 2017, 39(02):113-120.). The content is converted into mg/g. The total polyphenol content of Floccularia luteovirens from Qilian County of Qinghai Province is 7.67 (±0.06) mg/g, which is determined as the test example. The total polyphenol content of Floccularia luteovirens from Dangxiong County of Tibet Autonomous Region is 7.06 (±0.13) mg/g, which is determined as reference example 1. The total polyphenol content of Floccularia luteovirens from Shiqu County of Sichuan Province is 6.25 (±0.1) mg/g, which is determined as reference example 2 (see FIG. 1).

(2) The genome of Floccularia luteovirens samples is extracted by an Ezup column fungus genomic DNA extraction kit (No. B518259) of Sangon Biotech (Shanghai) Co., Ltd., and diluted to 20 ng/μL for fluorescent PCR amplification.

(3) Fluorescent PCR amplification of the SSR DNA barcode is carried out with primers in Table 1.

Fluorescent PCR amplification reaction system (10 μL): 5 μL of 2×Taq PCR MasterMix, 1 μL of template (genomic DNA), 0.1 μL of forward primer, 0.4 μL of reverse primer (concentrations of the forward primer and the reverse primer are both 10 uM), and 0.4 μL of M13 primer with fluorescence (concentration of 10 uM), wherein a volume is fixed to 10 μL with sterile deionized water.

Reaction conditions: performing pre-denaturation at 95° C. for 3 min, denaturation at 95° C. for 30 s, PCR annealing during the decrease from 62° C. to 55° C. for 30 s, and extension at 72° C. for 30 s, with a total of 10 cycles; performing denaturation at 95° C. for 30 s, annealing at 52° C. for 30 s, and extension at 72° C. for 30 s, with a total of 25 cycles; performing final extension at 72° C. for 20 min; and after heat preservation at 4° C. for 6 h, using the product for fluorescence capillary electrophoresis detection.

(4) After quantitative dilution of the PCR product, 1 μL of a PCR diluted products is denatured with 9 μL of formamide (including 1% internal standard), and then subjected to capillary fluorescence electrophoresis detection by a DNA sequencer ABI 3730×1. The internal standard, which is a molecular weight internal standard (also known as internal lane standard) LIZ-500 bp, is composed of 16 double-stranded DNA fragments labeled with LIZ fluorescein (orange), with molecular weights of 35, 50, 75, 100, 139, 150, 160, 200, 250, 300, 340, 350, 400, 450, 490 and 500 bp respectively. The fragment size in the amplification result electropherogram is equal to the actual bp number of the amplified fragment plus the M13 fluorescent primer (about 18 bp), with error of 1-2 bp. The amplified capillary electrophoresis peak is combined with the sequencing result; and the peak number indicates the number of heterozygous amplified fragments of the gene.

(5) The above methods are used to identify Floccularia luteovirens in test example, reference example 1 and reference example 2.

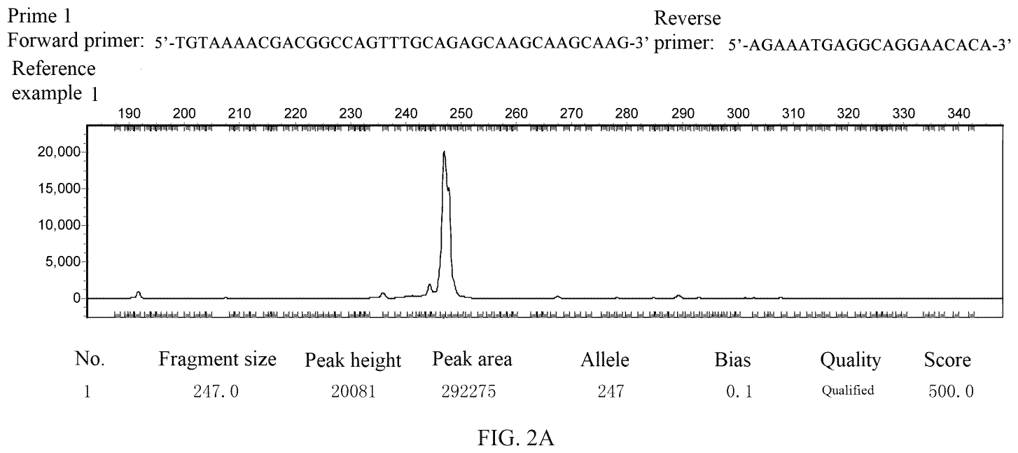

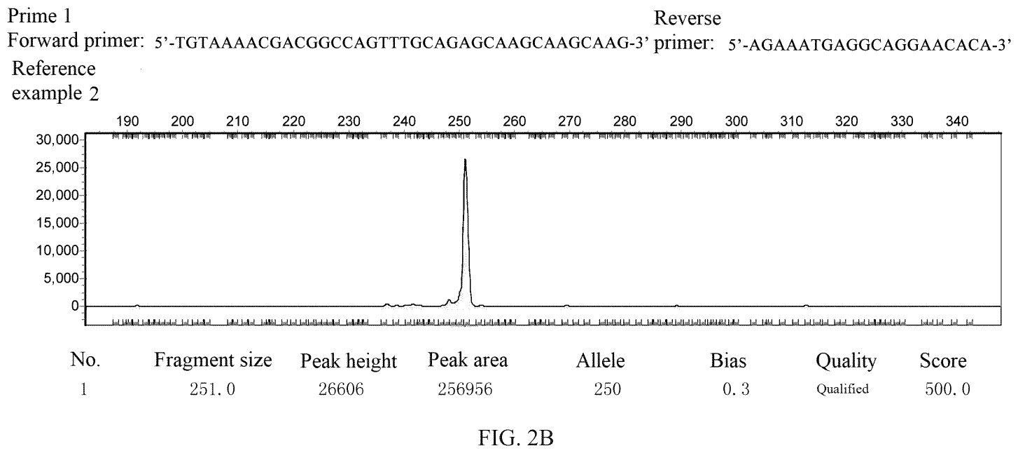

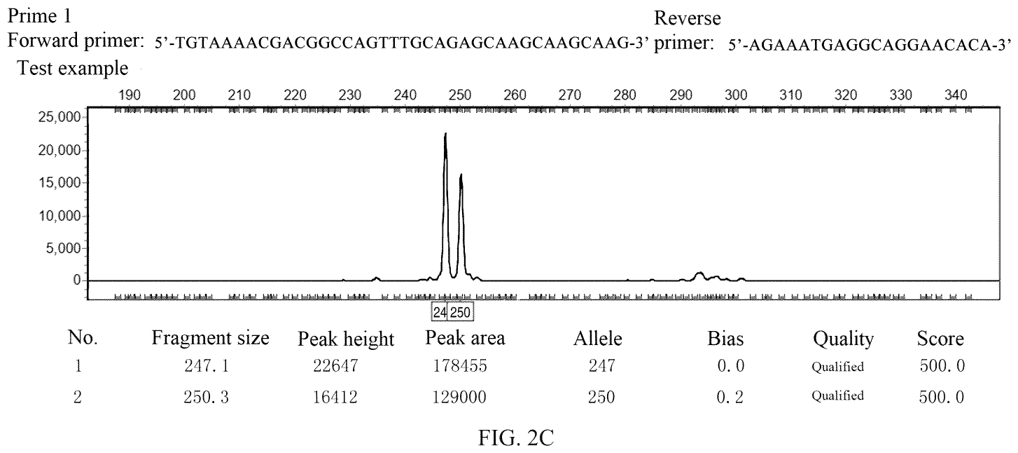

Amplification results of primer 1 are shown in FIGS. 2A-2C. When primer 1 is used for fluorescent PCR amplification, two fragments (two peaks) are obtained through amplification, which contain two SSR loci; and the SSR repetitive element is AAG. The amplified fragment obtained in the test example is characterized by containing a 229 bp fragment with 5 repeats and a 232 bp fragment with 6 repeats.

Amplified fragments of primer 1: (the statistical fragment length of electropherogram includes the M13 fluorescent primer; the specific sequence shows that the M13 fluorescent primer sequence (18 bp) is removed; and the underlined part is an SSR repetitive element.)

229 bp amplified fragment sequence:

(as shown in SEQ ID NO: 3)
TTGCAGAGCAAGCAAGCAAGTAGCAGACAAGTAAACCTTGAAC
AAGACTTGCAAATCACTATCACACAAAGCAACTACTACTACTA
CAAATCACTAATGAACAAGAAGAAGAAGAAGTAGAACAACAAG
TCTATGAAGAAGCACACAGTTACTGTTATGACCTTATCTGGAT
ACCATATATTTCTCTAGACTTTAGAATATATGTATAATGTGTT
CCTGCCTCATTTCT

232 bp amplified fragment sequence:

(as shown in SEQ ID NO: 4)
TTGCAGAGCAAGCAAGCAAGTAGCAGACAAGTAAACCTTGAAC
AAGACTTGCAAATCACTATCACACAAAGCAACTACTACTACTA
CAAATCACTAATGAACAAGAAGAAGAAGAAGAAGTAGAACAAC
AAGTCTATGAAGAAGCACACAGTTACTGTTATGACCTTATCTG
GATACCATATATTTCTCTAGACTTTAGAATATATGTATAATGT
GTTCCTGCCTCATTTCT.

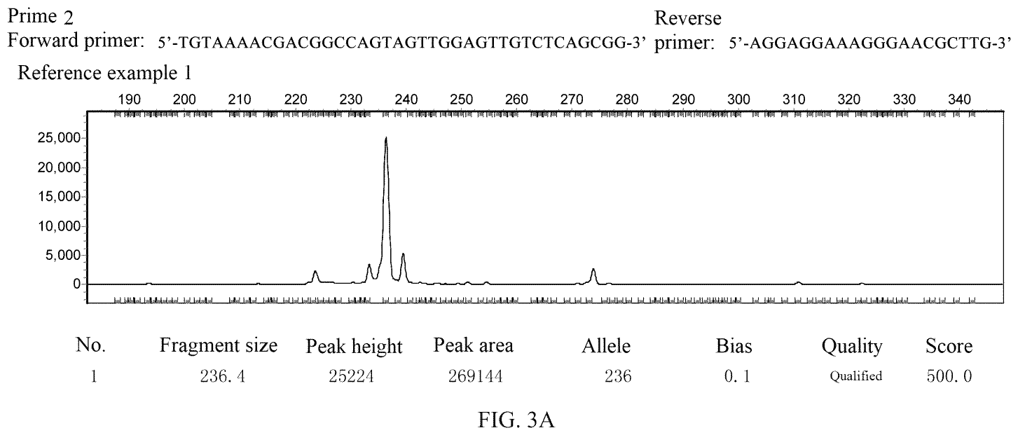

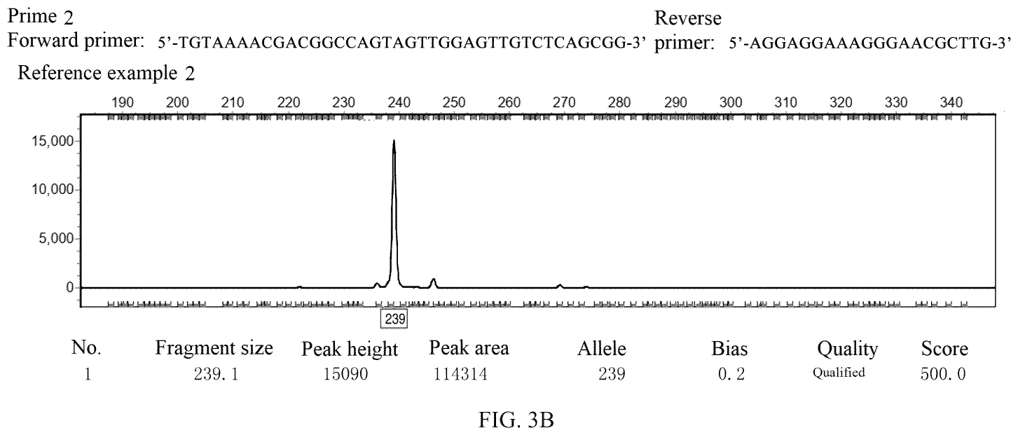

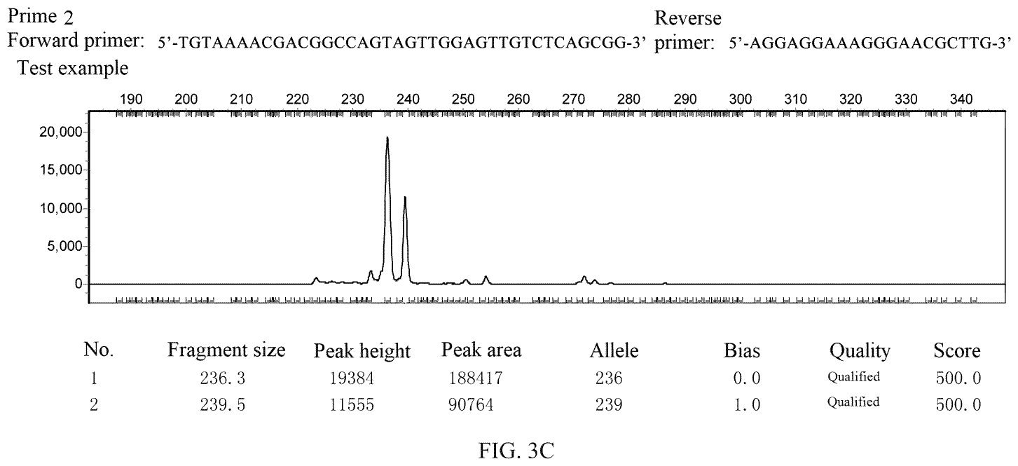

Amplification results of primer 2 are shown in FIGS. 3A-3C. When primer 2 is used for fluorescent PCR amplification, two fragments (two peaks) are obtained, which contain two SSR loci; and the SSR repetitive element is TCC. The amplified fragment obtained in the test example is characterized by containing a 218 bp fragment with 7 repeats and a 221 bp fragment with 8 repeats. In FIGS. 3A-3C, the weak signal hybrid peak during amplification of reference example 1 does not contain an SSR repetitive element.

Amplified fragments of primer 2: (the statistical fragment length of electropherogram includes the M13 fluorescent primer; the specific sequence shows that the M13 fluorescent primer sequence (18 bp) is removed; and the underlined part is an SSR repetitive element.)

218 bp amplified fragment sequence:

(as shown in SEQ ID NO: 7)
AGTTGGAGTTGTCTCAGCGGCGGCCATTGCTTTCGCAACCTCG
GCTTTCTGCTCCTCCTCGATACGAAGTCGTTCACGTTCCTCCT
CCTCCTCCTCCTCCTTCAGCCTCTCCTGCTCCTTGGCCTGCTC
TTCTGCGAGACGAACACGTTCTTTCTCTTCGTCTTCAAGCATA
CGTATACGTTCTTCCTCTTCTTTGCGCAAGCGTTCCCTTTCCT
CCT

221 bp amplified fragment sequence:

(as shown in SEQ ID NO: 8)
AGTTGGAGTTGTCTCAGCGGCGGCCATTGCTTTCGCAACCTCG
GCTTTCTGCTCCTCCTCGATACGAAGTCGTTCACGTTCCTCCT
CCTCCTCCTCCTCCTCCTTCAGCCTCTCCTGCTCCTTGGCCTG
CTCTTCTGCGAGACGAACACGTTCTTTCTCTTCGTCTTCAAGC
ATACGTATACGTTCTTCCTCTTCTTTGCGCAAGCGTTCCCTTT
CCTCCT

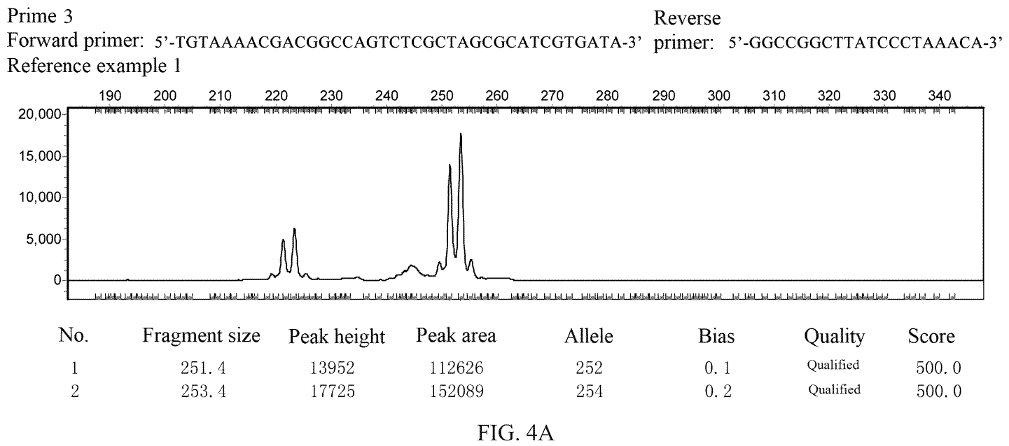

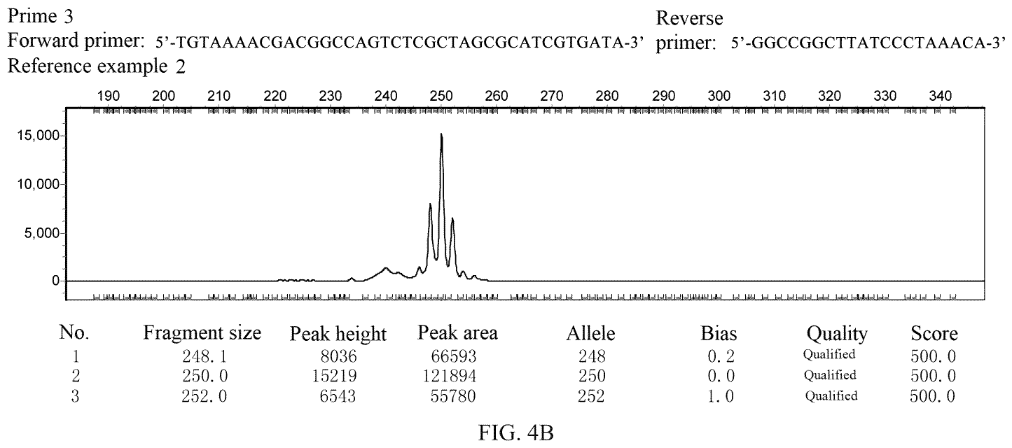

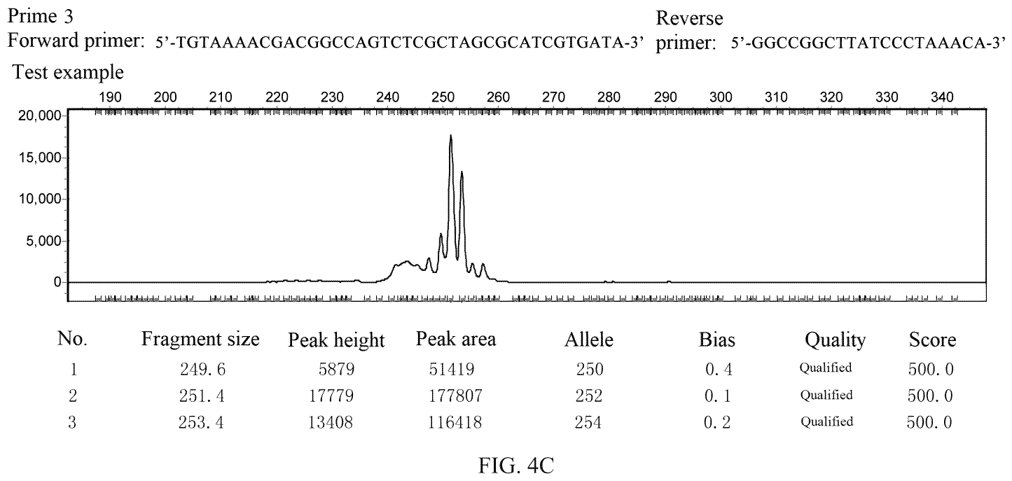

Amplification results of primer 3 are shown in FIGS. 4A-4C. When primer 3 is used for fluorescent PCR amplification, four fragments (four peaks) are obtained, which contain four SSR loci; and the SSR repetitive element is AT. The amplified fragment obtained in the test example is characterized by containing a 233 bp fragment with 8 repeats, a 235 bp fragment with 9 repeats and a 237 bp fragment with 10 repeats. In FIGS. 4A-4C, the amplified fragment of 223 bp belongs to nonspecific amplification; and in addition, the weak signal hybrid peak does not contain an SSR repetitive element.

Amplified fragments of primer 3: (the statistical fragment length of electropherogram includes the M13 fluorescent primer; the specific sequence shows that the M13 fluorescent primer sequence (17 bp) with the error of 1 bp is removed; and the underlined part is an SSR repetitive element.)

231 bp amplified fragment sequence:
(as shown in SEQ ID NO: 11)
CTCGCTAGCGCATCGTGATAAGAAAAGAGAGAGCTTAGAAAAG
AGGAAAAGTGACAAACGGAATGTAATGGTCGTGTTCGGATAAA
GTAGAGCATATACCTTCCCCACACCAGCCTCTTAAAACTAGTA
GTACTACACATCGCATGTAAAGCGCCCCAATGACCGCGTTTCT
GGATTTAAATATATACATATATATATATATAAATGGTCGTGTT
TAGGGATAAGCCGGCC
233 bp amplified fragment sequence:
(as shown in SEQ ID NO: 12)
CTCGCTAGCGCATCGTGATAAGAAAAGAGAGAGCTTAGAAAAG
AGGAAAAGTGACAAACGGAATGTAATGGTCGTGTTCGGATAAA
GTAGAGCATATACCTTCCCCACACCAGCCTCTTAAAACTAGTA
GTACTACACATCGCATGTAAAGCGCCCCAATGACCGCGTTTCT
GGATTTAAATATATACATATATATATATATATAAATGGTCGTG
TTTAGGGATAAGCCGGCC
235 bp amplified fragment sequence:
(as shown in SEQ ID NO: 13)
CTCGCTAGCGCATCGTGATAAGAAAAGAGAGAGCTTAGAAAAG
AGGAAAAGTGACAAACGGAATGTAATGGTCGTGTTCGGATAAA
GTAGAGCATATACCTTCCCCACACCAGCCTCTTAAAACTAGTA
GTACTACACATCGCATGTAAAGCGCCCCAATGACCGCGTTTCT
GGATTTAAATATATACATATATATATATATATATAAATGGTCG
TGTTTAGGGATAAGCCGGCC
237 bp amplified fragment sequence:
(as shown in SEQ ID NO: 14)
CTCGCTAGCGCATCGTGATAAGAAAAGAGAGAGCTTAGAAAAG
AGGAAAAGTGACAAACGGAATGTAATGGTCGTGTTCGGATAAA
GTAGAGCATATACCTTCCCCACACCAGCCTCTTAAAACTAGTA
GTACTACACATCGCATGTAAAGCGCCCCAATGACCGCGTTTCT
GGATTTAAATATATACATATATATATATATATATATAAATGGT
CGTGTTTAGGGATAAGCCGGCC

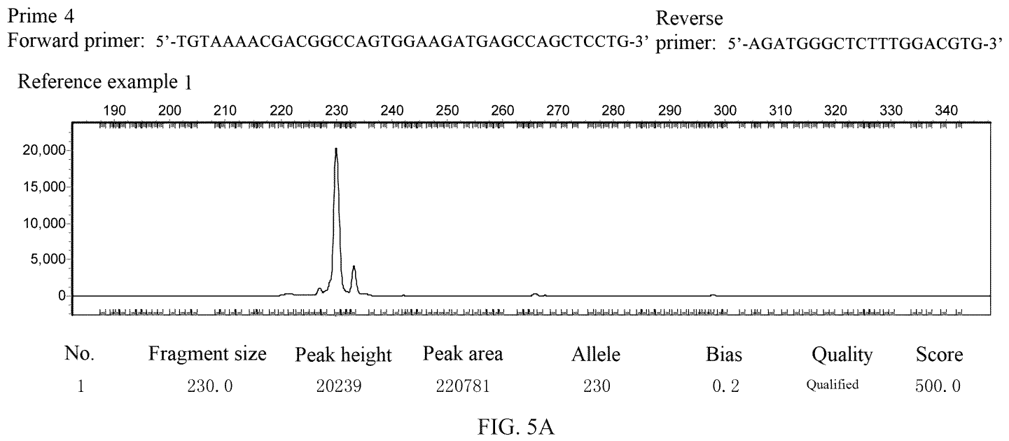

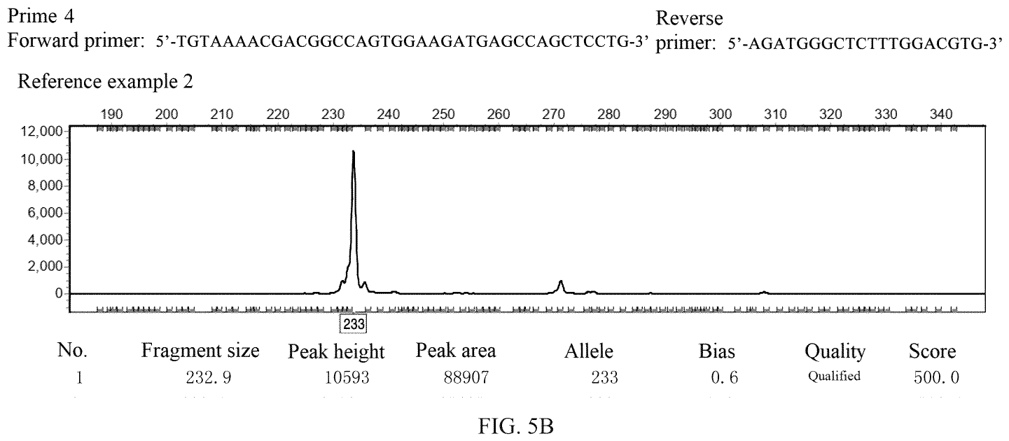

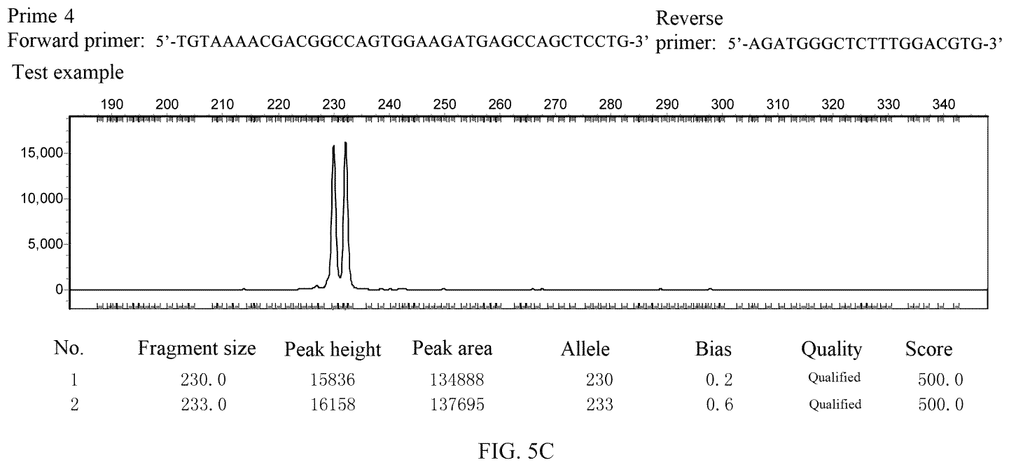

Amplification results of primer 4 are shown in FIGS. 5A-5C. When primer 4 is used for fluorescent PCR amplification, two fragments (two peaks) are obtained, which contain two SSR loci; and the SSR repetitive element is GCT. The amplified fragment obtained in the test example is characterized by containing a 212 bp fragment with 5 repeats of GCT and a 215 bp fragment with 6 repeats of GCT. In FIGS. 5A-5C, the weak signal hybrid peak during amplification of reference example 1 does not contain an SSR repetitive element.

Amplified fragments of primer 4: (the statistical fragment length of electropherogram includes the M13 fluorescent primer; the specific sequence shows that the M13 fluorescent primer sequence (18 bp); and the underlined part is an SSR repetitive element.)

212 bp amplified fragment sequence:
(as shown in SEQ ID NO: 17)
GGAAGATGAGCCAGCTCCTGCTGTCACTGAACCAGAGGCTGCT
GCTGCTGCTACTACTACCACTGAGACCCCAGCTGCCACCGAGG
AACCTTCTAAGGAGGAAGCTAAACCTGTATGTATACCCTAGCT
TTTGTGTTAACAACACTAATTCTTTTATGTTATAGGCTGCCGA
TTCTGACAATGGTAAGGCTGCACGTCCAAAGAGCCCATCT
215 bp amplified fragment sequence:
(as shown in SEQ ID NO: 18)
GGAAGATGAGCCAGCTCCTGCTGTCACTGAACCAGAGGCTGCT
GCTGCTGCTGCTACTACTACCACTGAGACCCCAGCTGCCACCG
AGGAACCTTCTAAGGAGGAAGCTAAACCTGTATGTATACCCTA
GCTTTTGTGTTAACAACACTAATTCTTTTATGTTATAGGCTGC
CGATTCTGACAATGGTAAGGCTGCACGTCCAAAGAGCCCATCT

Through comprehensive analysis of maps and sequencing results of test example, reference example 1 and reference example 2, the characteristic information of the DNA barcode of Floccularia luteovirens with the high total polyphenol content is shown in Table 2. Primer 1 carries out amplification to obtain the 229 bp fragment containing 5 AAG repetitive elements (as shown in SEQ ID NO:3) and the 232 bp fragment containing 6 AAG repetitive elements (as shown in SEQ ID NO:4); primer 2 carries out amplification to obtain the 218 bp fragment containing 7 TCC repetitive elements (as shown in SEQ ID NO:7) and the 221 bp fragment containing 8 TCC repetitive elements (as shown in SEQ ID NO:8); primer 3 carries out amplification to obtain the 233 bp fragment containing 8 AT repetitive elements (as shown in SEQ ID NO:12), the 235 bp fragment containing 9 AT repetitive elements (as shown in SEQ ID NO:13) and the 237 bp fragment containing 10 AT repetitive elements (as shown in SEQ ID NO:14); and primer 4 carries out amplification to obtain the 212 bp fragment containing 5 GCT repetitive elements (as shown in SEQ ID NO:17) and the 215 bp fragment containing 6 GCT repetitive elements (as shown in SEQ ID NO:18). Primers 1, 2, 3 and 4 or any combination of the primers can be used for comprehensive detection and judgment. When primers 1, 2, 3 and 4 are used together, the accuracy of screening the total polyphenol content index of Floccularia luteovirens is the best.

TABLE 2
DNA barcode characteristics of Floccularia luteovirens with high total
polyphenol content

| | Primer 1 | Primer 2 | Primer 3 | Primer 4 |
|---|---|---|---|---|
| Total | amplified | amplified | amplified | amplified |
| polyphenol | fragment | fragment | fragment | fragment |
| content | (SSR) | (SSR) | (SSR) | (SSR) |
| Reference | 229 bp (5AAG) | 218 bp (7TCC) | 235 bp (9AT) | 212 bp |
| example 1 | | | 237 bp (10AT) | (5GCT) |
| Low content | | | | |
| Reference | 232 bp (6AAG) | 221 bp (8TCC) | 231 bp (7AT) | 215 bp |
| example 2 | | | 233 bp (8AT) | (6GCT) |
| Low content | | | 235 bp (9AT) | |
| Test example | 229 bp (5AAG) | 218 bp (7TCC) | 233 bp (8AT) | 212 bp |
| High content | 232 bp (6AAG) | 221 bp (8TCC) | 235 bp (9AT) | (5GCT) |
| | | | 237 bp (10AT) | 215 bp |
| | | | | (6GCT) |

### Embodiment 3 Screening and Verification of a Total Polyphenol Content Index of Floccularia luteovirens

A DNA barcode of a total polyphenol content of Floccularia luteovirens is verified by blind testing.
Step 1, blind testing: taking samples from Qilian County of Qinghai Province with a total polyphenol content higher than or equal to 7.67 mg/g as a test group, taking samples from Dangxiong County of Tibet Autonomous Region and Shiqu County of Sichuan Province with total polyphenol contents lower than 91.100 (significance P<0.05) as a reference group 1 and a reference group 2, and taking 16 samples respectively, namely 48 samples in total, for blind testing;
step 2, testing: using primers (SEQ ID NO:1 and SEQ ID NO:2, SEQ TD NO:5 and SEQ ID NO: 6, SEQ ID NO:9 and SEQ ID NO: 10, and SEQ ID NO: 15 and SEQ TD NO: 16) for amplification and capillary electrophoresis. One or more pairs of primer groups can be combined for amplification, and the blind testing samples can be distinguished by DNA barcode characteristics of the test example;
step 3, unblinding: results are shown in Table 3. The unblinding results of 16 samples with high and low total polyphenol contents are all correct according to distinguishment by the DNA barcode characteristics of the total polyphenol content, indicating that the DNA barcode of the total polyphenol content is suitable for screening of the total polyphenol content character.

TABLE 3
Unblinding identification results based on DNA barcode
characteristics of total polyphenol content

| | Primer 1 | Primer 2 | Primer 3 | Primer 4 | | |
|---|---|---|---|---|---|---|
| Blind | amplified | amplified | amplified | amplified | | |
| testing | fragment | fragment | fragment | fragment | Unblinding | Distinguishment |
| No. | (SSR) | (SSR) | (SSR) | (SSR) | No. | results |
| 2, 4, | 229 bp | 218 bp | 235 bp | 229 bp | 33-48 | Total polyphenol |
| 5, 7, | (5AAG) | (7TCC) | (9AT) | (5AAG) | | content of |
| 8, 12, | | | 237 bp | | | reference group |
| 14, 15, | | | (10AT) | | | 1 is low |
| 19, 22, | | | | | | |
| 25, 26, | | | | | | |
| 34, 35, | | | | | | |
| 43, 45 | | | | | | |
| 1, 6, | 232 bp | 221 bp | 231 bp | 232 bp | 17-32 | Total polyphenol |
| 9, 13, | (6AAG) | (8TCC) | (7AT) | (6AAG) | | content of |
| 16, 17, | | | 233 bp | | | reference group |
| 20, 24, | | | (8AT) | | | 2 is low |
| 28, 30, | | | 235 bp | | | |
| 33, 38, | | | (9AT) | | | |
| 39, 42, | | | | | | |
| 46, 47 | | | | | | |
| 3, 10, | 229 bp | 218 bp | 233 bp | 229 bp | 1-16 | Total polyphenol |
| 11, 18, | (5AAG) | (7TCC) | (8AT) | (5AAG) | | content of test |
| 21, 23, | 232 bp | 221 bp | 235 bp | 232 bp | | group is high |
| 27, 29, | (6AAG) | (8TCC) | (9AT) | (6AAG) | | |
| 31, 32, | | | 237 bp | | | |
| 36, 37, | | | (10AT) | | | |
| 40, 41, | | | | | | |
| 44, 48 | | | | | | |

Each embodiment in the description is described in a progressive way. The difference of each embodiment from each other is the focus of explanation. The same and similar parts among all of the embodiments can be referred to each other.

The above description of the disclosed embodiments enables those skilled in the art to realize or use the present invention. Many modifications to these embodiments will be apparent to those skilled in the art. The general principle defined herein can be realized in other embodiments without departing from the spirit or scope of the present invention. Therefore, the present invention will not be limited to these embodiments shown herein, but will conform to the widest scope consistent with the principle and novel features disclosed herein.

What is claimed is:

1. A DNA barcode for screening a total polyphenol content index of Floccularia luteovirens, wherein a nucleotide sequence of the DNA barcode comprises one or more of:
SEQ ID NO:3,
and/or SEQ ID NO:4,
and/or a combination of SEQ ID NO:3 and SEQ ID NO:4,
and SEQ ID NO:7,
and/or SEQ ID NO:8,
and/or a combination of SEQ ID NO:7 and SEQ ID NO:8,
and/or a combination of SEQ ID NO:11 and SEQ ID NO:12 and SEQ ID NO:13,
and/or a combination of SEQ ID NO:12 and SEQ ID NO:13 and SEQ ID NO: 14,
and/or a combination of SEQ ID NO:13 and SEQ ID NO:14,
and/or SEQ ID NO:17,
and/or SEQ ID NO:18,
and/or a combination of SEQ ID NO:17 and SEQ ID NO:18.

2. A primer group for amplifying the DNA barcode for screening the total polyphenol content index of Floccularia luteovirens according to claim 1, wherein a nucleotide sequence of the primer group comprises one or more groups of:
SEQ ID NO: 1 and SEQ ID NO:2,
and/or SEQ ID NO:5 and SEQ ID NO:6,
and/or SEQ ID NO:9 and SEQ ID NO:10,
and/or SEQ ID NO:15 and SEQ ID NO:16.

3. The primer group according to claim 2, wherein the nucleotide sequence of the primer group comprises:
such as SEQ ID NO:1 and SEQ ID NO:2,
and SEQ ID NO:5 and SEQ ID NO:6,
and SEQ ID NO:9 and SEQ ID NO:10,
and SEQ ID NO:15 and SEQ ID NO:16.

4. A method for screening Floccularia luteovirens by using a total polyphenol content index, comprising the following steps:
S1, extracting genomic DNA of a sample to be tested;
S2, taking the genomic DNA in S1 as a template, and performing a fluorescent PCR amplification reaction on one or more of the primer groups according to claim 2 to obtain an amplification product;
S3, detecting the amplification product of S2 by capillary fluorescence electrophoresis, and judging by the number of fragments, the number of SSR loci, SSR repetitive elements and repeating times of the amplification product.

5. The method for screening Floccularia luteovirens by using the total polyphenol content index according to claim 4, wherein a judgment standard in step S3 is:
when the primer group of SEQ ID NO:1 and SEQ ID NO:2 is amplified to obtain a 229 bp fragment containing 5 AAG repetitive elements and a 232 bp fragment containing 6 AAG repetitive elements;
and/or the primer group of SEQ ID NO:5 and SEQ ID NO:6 is amplified to obtain a 218 bp fragment containing 7 TCC repetitive elements and a 221 bp fragment containing 8 TCC repetitive elements;
and/or the primer group of SEQ ID NO:9 and SEQ ID NO:10 is amplified to obtain a 233 bp fragment containing 8 AT repetitive elements, a 235 bp fragment containing 9 AT repetitive elements, and a 237 bp fragment containing 10 AT repetitive elements;
and/or the primer group of SEQ ID NO:15 and SEQ ID NO:16 is amplified to obtain a 212 bp fragment containing 5 GCT repetitive elements and a 215 bp fragment containing 6 GCT repetitive elements, the Floccularia luteovirens is determined as Floccularia luteovirens with high total polyphenol content.

6. The method for screening Floccularia luteovirens by using the total polyphenol content index according to claim 4, wherein a reaction system of the fluorescent PCR amplification reaction in step S2 is:
5 μL of 2×Taq PCR Master Mix, 1 μL of genomic DNA, 0.1 μL of forward primer, 0.4 μL of reverse primer, and 0.4 μL of M13 primer with fluorescence, wherein a volume is fixed to 10 μL with sterile deionized water.

7. The method for screening Floccularia luteovirens by using the total polyphenol content index according to claim 6, wherein the concentrations of the forward primer, the reverse primer and the M13 primer with fluorescence are all 10 uM.

8. The method for screening Floccularia luteovirens by using the total polyphenol content index according to claim 4, wherein a fluorescent PCR amplification reaction procedure in step S2 is:
performing pre-denaturation at 95° C. for 3 min, denaturation at 95° C. for 30 s, PCR annealing during the decrease from 62° C. to 55° C. for 30 s, and extension at 72° C. for 30 s, with a total of 10 cycles; performing denaturation at 95° C. for 30 s, annealing at 52° C. for 30 s, and extension at 72° C. for 30 s, with a total of 25 cycles; performing final extension at 72° C. for 20 min; and after heat preservation at 4° C. for 6 h, using the product for fluorescence capillary electrophoresis detection.

9. An application of the DNA barcode according to claim 1 in preparation of a product for screening high-quality Floccularia luteovirens with the total polyphenol content index.

10. An application of the primer group according to claim 2 in preparation of a product for screening high-quality Floccularia luteovirens with the total polyphenol content index.

11. A product for screening high-quality Floccularia luteovirens with the total polyphenol content index, comprising one or more of the primer groups according to claim 2, and satisfying the standard that the primer group of SEQ ID NO:1 and SEQ ID NO:2 is amplified to obtain a 229 bp fragment containing 5 AAG repetitive elements and a 232 bp fragment containing 6 AAG repetitive elements;
and/or the primer group of SEQ ID NO:5 and SEQ ID NO:6 is amplified to obtain a 218 bp fragment containing 7 TCC repetitive elements and a 221 bp fragment containing 8 TCC repetitive elements;
and/or the primer group of SEQ ID NO:9 and SEQ ID NO:10 is amplified to obtain a 233 bp fragment containing 8 AT repetitive elements, a 235 bp fragment containing 9AT repetitive elements and a 237 bp fragment containing 10 AT repetitive elements;
and/or the primer group of SEQ ID NO:15 and SEQ ID NO:16 is amplified to obtain a 212 bp fragment containing 5 GCT repetitive elements and a 215 bp fragment containing 6 GCT repetitive elements.
